(19) United States
(12) Patent Application Publication
(10) Pub. No.: US 2006/0074481 A1
(43) Pub. Date: Apr. 6, 2006

(54) Graft including expandable cuff

(76) Inventors: Gil Vardi, St. Louis, MO (US); Harvey Serota, St. Louis, MO (US)

Correspondence Address: PATRICK W. RASCHE;ARMSTRONG TEASDALE LLP, ONE METROPOLITAN SQUARE, SUITE 2600, ST. LOUIS, MO, 63102-2740, US

(21) Appl. No.: 10/958,119

(22) Filed: Oct. 4, 2004

(51) Int. Cl. A61F 2/06

(52) U.S. Cl. 623/1.36

(57) ABSTRACT

A graft assembly includes a first graft having first and second ends and a second graft having first and second ends. In addition, the graft assembly includes a first cuff attached to the first end of the first graft and a second cuff attached to the first end of the second graft. Each cuff is configured to expand and exert a radially outward force against the other cuff and a vessel wall.

### BACKGROUND OF THE INVENTION

This invention relates generally to treatment of an aneurysm in a blood vessel, and more specifically to methods and apparatus for a graft assembly.

A stent is an elongated tubular wire frame device providing structural support for a vessel wall. A graft is an elongated tubular device through which blood may flow. The combination of stent and graft devices is known as a stent graft. Stent grafts may be used to treat aneurysms in the vascular system. An aneurysm is a degeneration of the vessel wall whereby the wall weakens and balloons outward by thinning. Left untreated, an aneurysm can lead to rupture causing fatal hemorrhaging.

The traditional method of treating an aneurysm involving a large vessel, such as an abdominal aortic aneurysm, is by an invasive surgical repair procedure. The surgery requires a significant abdominal incision so that the graft may be implanted directly in the affected area. The patient is placed under general anesthesia and requires a significant amount of time in an intensive care unit following the procedure for post-operative recovery.

Due to the complexities of surgical repair, alternative approaches have been developed to deploy a stent graft endoluminally. Past approaches have included the introduction of multiple stent grafts that are expandable by a balloon catheter or are self-expanding. In addition, single stent grafts have been employed that include multiple branches. A problem with the existing stent graft configurations is the difficulty of treating aneurysms located near a bifurcation in the vasculature. Another problem is the insertion of large profile devices designed to fit within the aorta. The large profile requires a surgical incision for insertion.

### BRIEF DESCRIPTION OF THE INVENTION

In one aspect, a graft assembly is provided that includes a first graft having first and second ends and a second graft having first and second ends. In addition, the graft assembly includes a first cuff attached to the first end of the first graft and a second cuff attached to the first end of the second graft. Each cuff is configured to expand and exert a radially outward force against the other cuff and a vessel wall.

In another aspect, an endoluminal stent graft prosthesis is provided for a bifurcated blood vessel. The stent graft prosthesis includes a first stent graft having first and second ends. The first end of the first stent graft is configured to extend into a first branch of a branched vessel. The stent graft prosthesis also includes a second stent graft having first and second ends. The first end of the second stent graft is configured to extend into a second branch of the branched vessel. The stent graft prosthesis also includes a first cuff attached to the second end of the first stent graft and a second cuff attached to the second end of the second stent graft. Each cuff is configured to expand and exert a radially outward force against the other cuff and a vessel wall.

In another aspect, a method for treating a deformity in the wall of a blood vessel is provided. The method includes introducing a first stent graft through a first access site, the first stent graft including a proximal end, a distal end and an expandable cuff attached to the distal end. The method also including introducing a second stent graft through a second access site, the second stent graft including a proximal end, a distal end and an expandable cuff attached to the distal end. The method further including advancing the first and second stent grafts until at least a portion of each of the stent grafts extends across the deformity in the vessel wall and expanding the expandable cuffs of the first and second stent grafts to form a seal between the other cuff and the vessel wall.

In another aspect, a stent graft is provided that includes first and second ends, a first cuff located at the first end, and a second cuff located at the second end. Each cuff is configured to expand and exert a radially outward force against a vessel wall.

In another aspect, a stent graft is provided that includes first and second ends and a first cuff located at the first end. The cuff is configured to expand and exert a radially outward force against a vessel wall. The cuff includes an inflation port including a valve configured to seal the cuff after an inflation tube is removed therefrom.

In another aspect, a stent graft is provided that includes first and second ends and a first cuff located at the first end. The cuff is configured to expand and exert a radially outward force against a vessel wall. The cuff includes an inflation tube including a weakened portion that can be severed more easily than the remaining portion of the inflation tube.

In another aspect, a stent graft is provided that includes first and second ends and a first cuff located at the first end. The cuff is configured to expand and exert a radially outward force against a vessel wall. The cuff includes a sponge material configured to expand upon exposure to moisture.

### BRIEF DESCRIPTION OF THE DRAWINGS

FIG. 1 shows a plan view of a stent graft assembly in accordance with one embodiment of the present invention;

FIG. 2 is a cross-sectional view taken along line 1-1 of FIG. 1.

FIG. 3 shows a partial cross-sectional view of an aneurysm in the process of being repaired in accordance with one embodiment of the present invention;

FIG. 4 is a schematic view of a stent graft including a perforated inflation tube;

FIG. 5 is a schematic view of a stent graft including an inflation port;

FIG. 6 is a schematic view of a stent graft including a sponge material;

FIG. 7 is a schematic view of a stent graft including two expandable cuffs;

FIG. 8 is a schematic view of a stent graft including a cuff that extends substantially the entire length of the graft; and

FIG. 9 is a schematic view of a stent graft that includes four cuffs attached to the graft.

FIG. 10 shows a plan view of a stent graft assembly in accordance with another embodiment of the present invention;

FIG. 11 is a cross-sectional view taken along line 2-2 of FIG. 10.

### DETAILED DESCRIPTION OF THE INVENTION

Exemplary embodiments of stent grafts are described below. In one embodiment, a stent graft assembly includes at least one stent graft having an expandable cuff at one end. A second stent graft may be employed at the same location to accommodate a branched artery or a larger size than can be percutaneously inserted. In one embodiment the cuff is inflatable, while in an alternative embodiment, the cuff includes a sponge material that expands upon exposure to moisture. In a further embodiment, the stent graft includes a first cuff located at the distal end and a second cuff located at the proximal end. In another embodiment, each stent graft has a flattened side and when the stent grafts are placed within a vessel

The methods and apparatus for a stent graft described herein are illustrated with reference to the figures wherein similar numbers indicate the same elements in all figures. Such figures are intended to be illustrative rather than limiting and are included herewith to facilitate explanation of an exemplary embodiment of the stent graft.

The terms distal and proximal as used herein refer to the orientation of the stent graft within the body of a patient. As used herein, distal refers to that end of the stent graft extended farthest into the body while the term proximal refers to that end of the stent graft located farthest from the distal end of the stent graft.

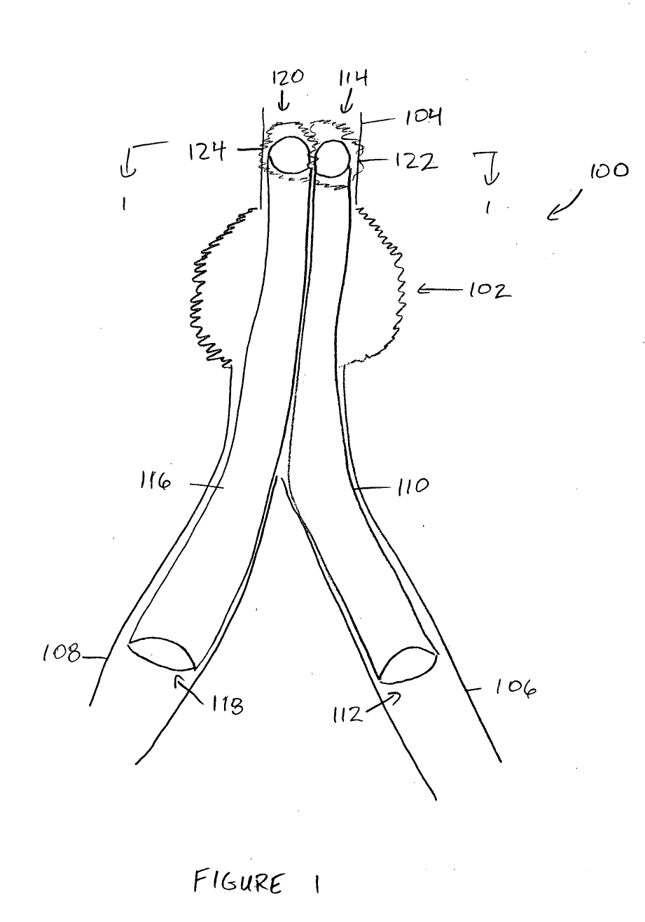

FIG. 1 shows a plan view of a stent graft assembly 100. In the exemplary embodiment, an aneurysm 102 is an abdominal aortic aneurysm in an aorta 104 that has common iliac arteries 106 and 108. The invention is not limited to the repair of abdominal aortic aneurysms. For example, the invention may be used in the thoracic aorta to repair thoracic aortic aneurysms. Furthermore, the invention may be used in a variety of body lumen (either bifurcated or non-bifurcated) where stent grafts are inserted.

In the exemplary embodiment, a first stent graft 110 includes a proximal end 112 and a distal end 114 and a second stent graft 116 includes a proximal end 118 and a distal end 120. An expandable cuff 122 is attached to distal end 114 of stent graft 110 and an expandable cuff 124 is attached to distal end 120 of stent graft 116. Stent grafts 110 and 116 have a generally circular cross-sectional configuration. Cuffs 122, 124 may be expanded with a fluid and inflated to a specific expanded configuration. Alternatively, cuffs 122, 124 may comprise a sponge material that expands upon exposure to moisture. In one embodiment, cuffs 122, 124 have a “D” shape in the expanded configuration. Alternatively, cuffs 122, 124 have a substantially spherical or cylindrical shape in the expanded configuration, but due to the pressure applied to the adjacent cuff, each cuff conforms to a “D” shape when expanded in the vessel due to space constraints

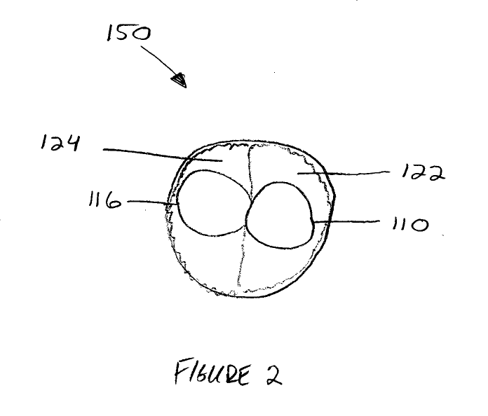

FIG. 2 is a cross-sectional view 150 taken along line 1-1 of FIG. 1. Expandable cuffs 122 and 124 are shown in their expanded state. Each of cuffs 122, 124 have a “D” configuration when expanded while the cross-sections of stent grafts 110 and 116 remain substantially circular.

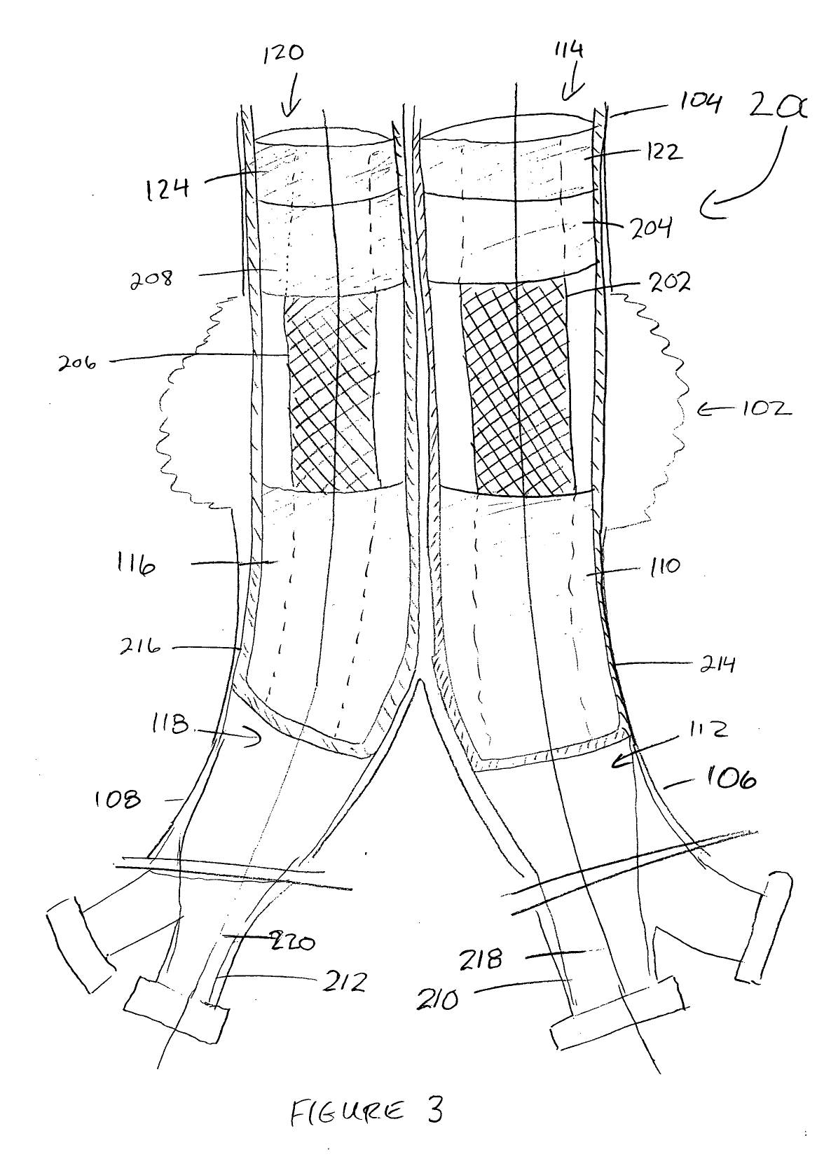

FIG. 3 shows a partial cross-sectional view of aneurysm 102 in the process of being repaired by stent graft assembly 200. Stent graft 110 is a composite device including a stent 202 and a graft 204, and stent graft 116 is a composite device including a stent 206 and a graft 208.

Stents 202 and 206 are elongated tubular wire frame devices manufactured from one or more of a variety of materials providing sufficient structural support and biocompatibility to allow for the treatment of a weakened or diseased vessel wall. Examples of suitable materials include stainless steel and nitinol. Grafts 204 and 208 are elongated tubular devices through which blood may flow. Grafts 204 and 208 are manufactured from one or more of a variety of materials providing sufficient mechanical properties for allowing the flow of blood and biocompatibility. Examples of suitable materials include DACRON® (polyethylene terephthalate) and TEFLON® (polytetrafluoroethylene).

In one embodiment, inflatable cuffs 122 and 124 are manufactured from one or more of a variety of materials allowing for a radially outward force to be exerted against the other of the cuffs and a vessel wall. A suitable material for the fabrication of inflatable cuffs 122 and 124 include a compliant material such as latex. An alternative material for the fabrication of inflatable cuffs 122 and 124 include a non-compliant material such as nylon.

In one embodiment, expandable cuffs 122, 124 are fabricated from a sponge material. The material is at least one of a natural sponge material and a synthetic absorbent material that functions as a sponge. In the example embodiment, the sponge material includes a thrombogenic material. For example, the sponge material is soaked with a pro-coagulent. Upon exposure to moisture, e.g., the patient's blood, the moisture is absorbed by the sponge material, causing the cuff to expand. The blood reacts with the thrombogenic material and causes the blood to clot in the expanded cuff and harden in the expanded shape.

In the example embodiment, stent graft 110 and stent graft 116 are delivered by catheters. A first introducer delivery device 210 and a second introducer delivery device 212, both including a tubular sheath, are inserted into the patient's vasculature through the femoral artery by means of a femoral arteriotomy or percutaneous delivery. First delivery catheter 214 and second delivery catheter 216 are then fed into the vasculature by means of these introducers. A first guide wire 218 is advanced through the femoral artery, external iliac artery, common iliac artery 106, and aneurysm 102 until it extends into aorta 104. A second guide wire 220 is advanced through the femoral artery, external iliac artery, common iliac artery 108, and aneurysm 102 until it also extends into aorta 104. First delivery catheter 214 and second delivery catheter 216 are guided by means of first guide wire 218 and second guide wire 220 until each extend across aneurysm 102.

Stent graft 110 is introduced using first delivery catheter 214 and stent graft 116 is introduced using second delivery catheter 216 until at least a portion of distal end 114 of stent graft 110 and distal end 120 of stent graft 116 extend across aneurysm 102 and are aligned with each other. In one embodiment, the alignment of stent grafts 110 and 116 is monitored with the use of radio-opaque markers.

Cuff 122 is expanded to exert a radially outward force against cuff 124 and the vessel wall. Cuff 124 is expanded to exert a radially outward force against cuff 122 and the vessel wall. Cuffs 122 and 124 may be expanded either simultaneously or sequentially. In one embodiment, cuffs 122 and 124 are inflated with a variety of materials that promote a seal between inflatable cuffs 122, 124 and the vessel wall. In one example, inflatable cuffs 122 and 124 are inflated with a hardening agent, such as collagen or a mixture of thrombin and the patient's blood. After inflation, the material hardens and the cuff maintains its expanded shape even if the integrity of the cuff is compromised. In another example, inflatable cuffs 122, 124 are inflated with a synthetic material such as an epoxy that hardens upon inflation of cuffs 122, 124 and maintains the expanded cuff shape even if the integrity of the cuff is compromised. In either example, cuffs 122, 124 are inflated to form a seal between the stent graft and the vessel wall even if the integrity of a cuff is compromised. In another embodiment, inflatable cuffs 122 and 124 are inflated with a saline solution, allowing for easy deflation and retrieval of stent graft 110. At the completion of the delivery procedure, the delivery devices are removed and any incisions are closed by known techniques such as applying pressure to stop the bleeding, suturing by standard vascular surgical techniques, and utilizing a known closure device.

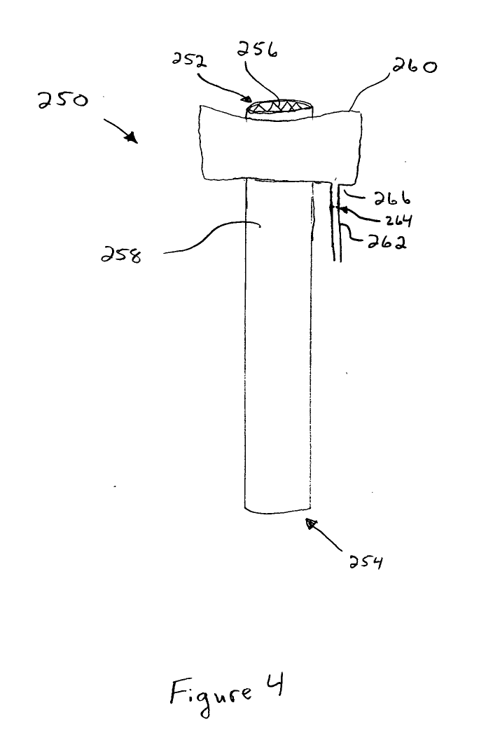

FIG. 4 is a schematic view of a stent graft 250 including a distal end 252, a proximal end 254, a stent 256, a graft 258 and a cuff 260. An inflation tube 262 extends from cuff 260 and is used to provide inflation fluid to cuff 260. In one embodiment, inflation tube 262 includes a weakened section 264 or a closure device near cuff end 266. Weakened section 264 is, in one embodiment, a perforated section configured to sever and allow inflation tube 262 to separate. Weakened section 264 is configured to provide a release mechanism of inflation tube 262 from cuff 260. Weakened section 264 has sufficient strength to enable tube 262 to provide enough fluid to cuff 260 such that cuff 260 inflates to the desired size and shape. In addition, weakened section 264 is configured to sever when a sufficient stress is applied to tube 262. Such stress is applied after cuff 260 has been adequately inflated and as tube 262 is pulled away from stent graft 250. In one embodiment, this stress is a pressure less than 5 atmospheres. In another embodiment, this stress is a pressure of about 1-2 atmospheres. In one embodiment, tube 262 is attached to a delivery mechanism, such as a delivery catheter, and when the delivery catheter is removed inflation tube 262 is severed at weakened section 264. In the exemplary embodiment, tube 262 is severed after cuff 260 is inflated and hardened such that cuff 260 retains its expanded configuration even upon severance of tube 262 and hence the loss of integrity of cuff 260.

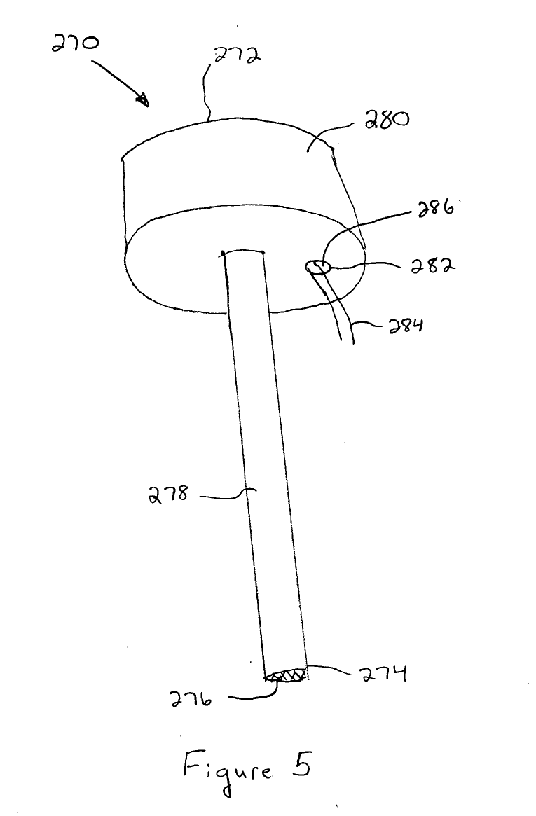

FIG. 5 illustrates an alternative embodiment of a stent graft 270 including a distal end 272, a proximal end 274, a stent 276, a graft 278 and a cuff 280. Cuff 280 includes an inflation port 282 configured to accept and release an inflation tube 284. In one embodiment, inflation port 282 includes a valve 284 configured to prevent fluid to flow out of cuff 280 after cuff 280 is inflated and tube 284 is removed from inflation port 282. In the exemplary embodiment, valve 286 is a flap valve in which the flap is a compliant member, although other types of valves can be used as long as they provide a seal sufficient to maintain cuff 280 in the expanded configuration. Valve 286 is configured to remain in the sealed position after removal of tube 284. In the exemplary embodiment, tube 284 is inserted within valve 284 prior to insertion of stent graft 270 into the body. After appropriate positioning and expansion of stent graft 270 within a vessel, cuff 280 is inflated with a fluid that passes through inflation tube 284. The delivery catheter is then removed along with inflation tube 284. Upon removal of inflation tube from inflation port 282, valve 286 seals and prevents fluid from escaping from expanded cuff 280. Alternatively, inflation tube 284 remains within inflation port 282 until the inflation media within cuff 280 hardens such that cuff 280 remains in the expanded configuration. Inflation tube 284 is then removed from inflation port 282 without the contents within cuff 280 escaping into the lumen.

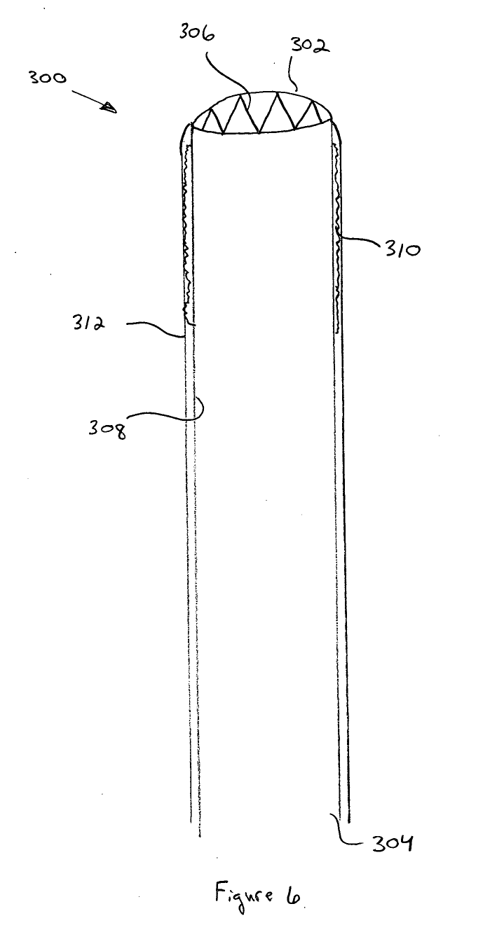

FIG. 6 is a schematic view of a stent graft 300 including a distal end 302, a proximal end 304, a stent 306, a graft 308 and a cuff 310. Cuff 310 is fabricated from a sponge material that expands upon absorption of liquid. Accordingly, during insertion of stent graft 300 into a body, cuff 310 is covered with a shield 312. After stent graft 300 is located at the appropriate location, shield 310 is removed and the sponge material of cuff 310 is exposed to the patient's blood. In one embodiment, shield 312 is a porous structure. In another embodiment, shield 312 is a non-porous structure.

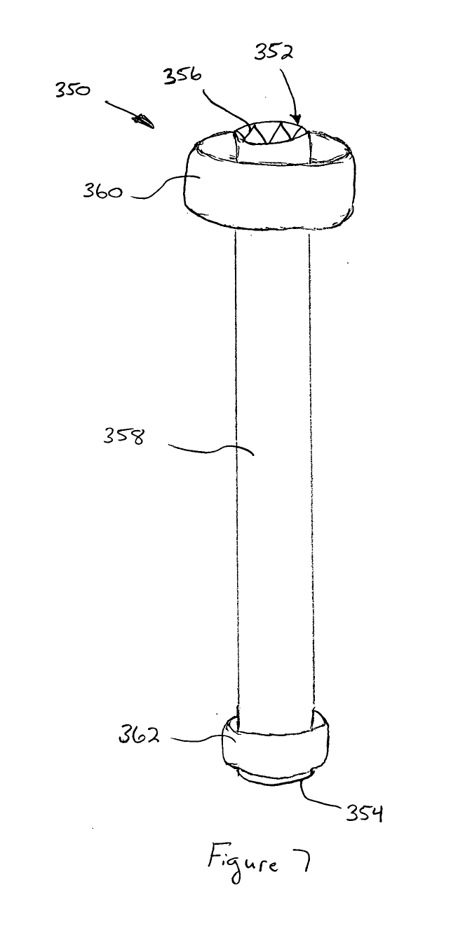

FIG. 7 illustrates a stent graft 350 including a distal end 352, a proximal end 354, a stent 356, a graft 358, a distal cuff 360, and a proximal cuff 362. Distal cuff 360 is configured to seal a large lumen, such as the aorta, either alone or in combination with a second stent graft 350 and proximal cuff 362 is configured to seal a smaller lumen, such as a common iliac artery. In one embodiment, cuffs 360, 362 are inflatable cuffs and use at least one of an inflation port with a valve and a severable inflation tube. In another embodiment, cuffs 360, 362 are fabricated from a sponge material that expands upon exposure to moisture. In a further embodiment, one of cuffs 360, 362 is an inflatable cuff while the other of cuffs 360, 362 is fabricated from a sponge material.

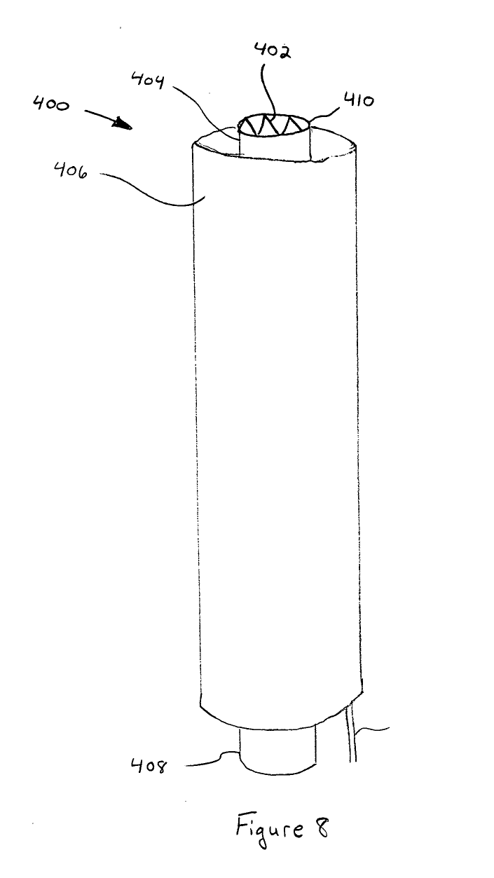

FIG. 8 illustrates a stent graft 400 including a stent 402, a graft 404 and a cuff 406. Graft 400 includes a proximal end 408 and a distal end 410. Cuff 406 extends substantially the entire length of stent graft 400. In one embodiment, cuff 406 extends from within half an inch of proximal end 408 to within half an inch of distal end 410. Cuff 406 includes an inflation tube 408 used to inflate cuff 406 with a fluid.

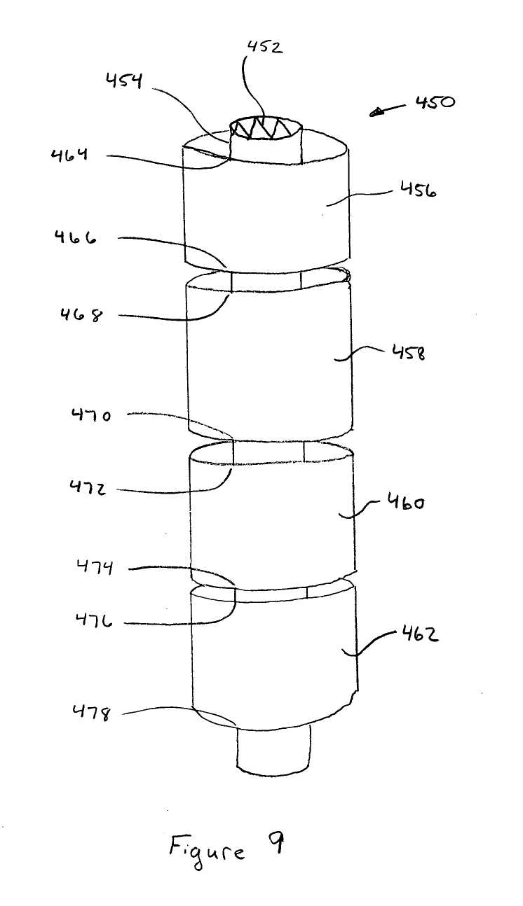

FIG. 9 illustrates a stent graft 450 including a stent 452, a graft 454, a first cuff 456, a second cuff 458, a third cuff 460, and a fourth cuff 462. First cuff 456 includes a distal end 464 and a proximal end 466 and cuff 456 is attached to graft 454 at distal end 464. Second cuff 458 includes a distal end 468 and a proximal end 470 and cuff 458 is attached to graft 454 at distal end 470. Third cuff 460 includes a distal end 472 and a proximal end 474 and cuff 460 is attached to graft 454 at distal end 472. Fourth cuff 462 includes a distal end 476 and a proximal end 478 and cuff 462 is attached to graft 454 at distal end 476. In one embodiment, cuffs 456, 458, 460, and 462 are attached to graft 454 at only distal ends 464, 468, 472, and 476. In another embodiment, cuffs 456, 458, 460, and 462 are attached to graft 454 along their entire length. In a further embodiment, cuffs 456, 458, 460, and 462 are attached to graft 454 along a distal portion of cuffs 456, 458, 460, and 462 that extends to substantially a middle of each of cuffs 456, 458, 460, and 462 to form a skirt around graft 454 when cuffs 456, 458, 460, and 462 are expanded. In the embodiment shown in FIG. 9, cuffs 456, 458, 460, and 462 comprise a sponge material. Alternatively, cuffs 456, 458, 460, and 462 are inflatable members and an inflation tube extends between adjacent cuffs.

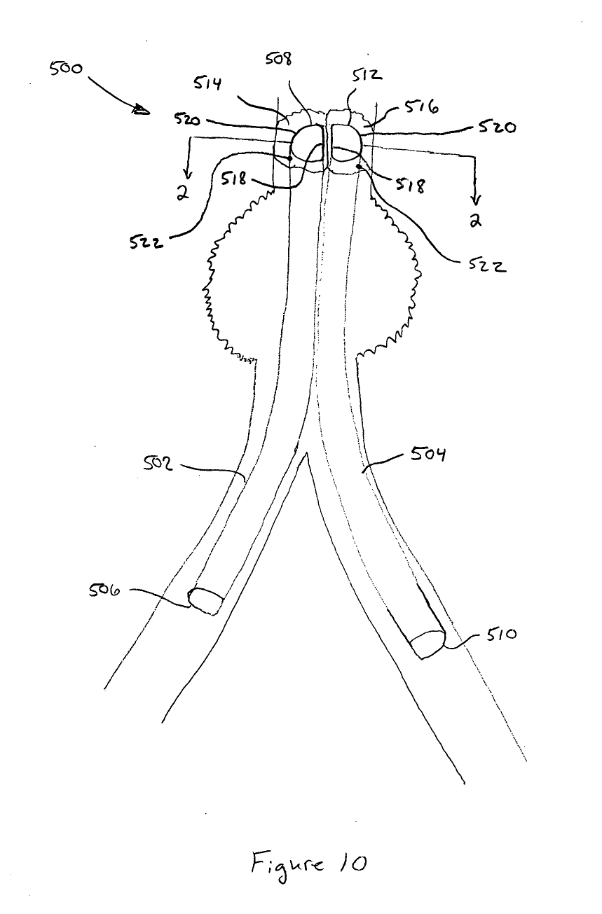

FIG. 10 is a plan view of a stent graft assembly 500 including a first stent graft 502 and a second stent graft 504. First stent graft 502 includes a proximal end 506 and a distal end 508 and second stent graft 504 includes a proximal end 510 and a distal end 512. An expandable cuff 514 is attached to distal end 508 of stent graft 502 and an expandable cuff 516 is attached to distal end 512 of stent graft 504. Stent grafts 502 and 504 have a generally “D” shaped cross-sectional configuration, for example a flattened or straight portion 518 attached to an arcuate or substantially semi-circular portion 520. Stent grafts 502 and 504 each include a radiopaque marker 522 attached to one side thereof. Radiopaque markers 522 are utilized to properly align stent grafts 502 and 504 during delivery such that flattened sides 518 are adjacent each other after stent grafts 502 and 504 have been inserted within the vessel.

Cuffs 514, 516 may be expanded with a fluid and inflated to a specific expanded configuration. Alternatively, cuffs 514, 516 may comprise a sponge material that expands upon exposure to moisture. Cuffs 514, 516 each have a “D” configuration (similar to the configuration of stent grafts 502, 504) when in the expanded configuration. Alternatively, cuffs 514, 516 have a substantially spherical or cylindrical shape in the expanded configuration, but due to the pressure applied to the adjacent cuff, each cuff conforms to a “D” shape when expanded in the vessel due to space constraints. In one embodiment, stent grafts 502 and 504 do not contact each other and a space extends between stent grafts 502 and 504 at distal ends 508 and 512. Cuffs 514 and 516 extend within the space and contact each other when stent grafts 502 and 504 are properly positioned within a vessel. In another embodiment, stent grafts 502 and 504 contact each other along flattened side 518 and cuffs 514 and 516 prevent fluid flowing between cuffs 502 and 504.

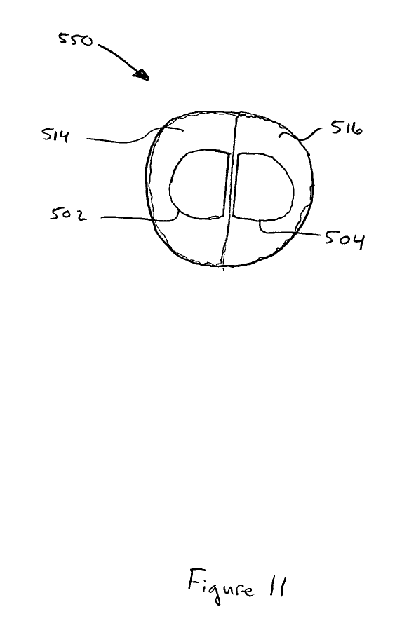

FIG. 11 is a cross-sectional view 550 taken along line 2-2 of FIG. 10. Expandable cuffs 514, 516 are shown in their expanded state. Each of cuffs 514, 516 have a “D” configuration when expanded and the cross sections of each of stent grafts 502, 504 also have a “D” configuration.

Although stent grafts are described hereafter, it is to be understood that grafts could utilize the same technology without being attached to a stent. While the invention has been described in terms of various specific embodiments, those skilled in the art will recognize that the invention can be practiced with modification within the spirit and scope of the claims.

What is claimed is:

1. A graft assembly comprising:
a first graft having first and second ends;
a second graft having first and second ends; and
a first cuff attached to said first end of said first graft and a second cuff attached to said first end of said second graft, each said cuff configured to expand and exert a radially outward force against the other said cuff and a vessel wall.

2. The graft assembly of claim 1 wherein said cuffs are inflatable with a fluid.

3. The graft assembly of claim 2 wherein the fluid comprises at least one of thrombin and collagen.

4. The graft assembly of claim 3 wherein the fluid further comprises the patient's blood.

5. The graft assembly of claim 1 wherein each said cuff has a “D” configuration when expanded.

6. The graft assembly of claim 1 wherein said grafts are positioned in a vessel by percutaneous delivery.

7. The graft assembly of claim 1 wherein each said cuff further comprises an inflation tube extending therefrom.

8. The graft assembly of claim 6 wherein each said inflation tube has a weakened portion that can be severed more easily than the remaining portion of said inflation tube.

9. The graft assembly of claim 1 wherein each said cuff further comprises an inflation port comprising a valve configured to seal a respective said cuff upon removal of a respective inflation tube.

10. The graft assembly of claim 1 wherein said cuffs comprise a sponge material that expands upon exposure to moisture.

11. The graft assembly of claim 1 further comprising a first stent positioned within said first graft and a second stent positioned within said second graft.

12. The graft assembly of claim 1 further comprising a third cuff attached to a middle portion of said first graft and a fourth cuff attached to a middle portion of said second graft.

13. The graft assembly of claim 1 wherein each said cuff is attached to a respective said graft at only a distal end of said cuff.

14. The graft assembly of claim 1 wherein each said cuff extends substantially an entire length of a respective said graft.

15. The stent graft assembly of claim 1 wherein each said stent graft has a “D” shaped cross-sectional configuration.

16. The stent graft assembly of claim 1 wherein each said stent graft further comprises a radiopaque marker.

17. An endoluminal stent graft prosthesis for a bifurcated blood vessel comprising:
a first stent graft having first and second ends, said first end of said first stent graft configured to extend into a first branch of a branched vessel;
a second stent graft having first and second ends, said first end of said second stent graft configured to extend into a second branch of a branched vessel; and
a first cuff attached to said second end of said first stent graft and a second cuff attached to said second end of said second stent graft, each said cuff configured to expand and exert a radially outward force against the other said cuff and a vessel wall.

18. The endoluminal stent graft prosthesis of claim 17 wherein said cuffs are inflatable with a fluid.

19. The endoluminal stent graft prosthesis of claim 17 wherein the inflation fluid includes at least one of a material comprising collagen and a material comprising a mixture of thrombin and the patient's blood.

20. The endoluminal stent graft prosthesis of claim 17 wherein each said cuff comprises a “D” configuration after expansion.

21. The endoluminal stent graft prosthesis of claim 17 wherein said stent grafts are positioned in a vessel by percutaneous delivery.

22. The endoluminal stent graft prosthesis of claim 17 wherein each said cuff further comprises an inflation tube extending therefrom.

23. The endoluminal stent graft prosthesis of claim 22 wherein said inflation tube has a weakened portion that can be severed more easily than the remaining portion of said inflation tube.

24. The endoluminal stent graft prosthesis of claim 17 wherein said cuffs further comprise an inflation port comprising a valve configured to seal said cuff upon removal of an inflation tube.

25. The endoluminal stent graft prosthesis of claim 17 wherein said cuffs comprise a sponge material that expands upon exposure to moisture.

26. A method for treating a deformity in the wall of a blood vessel, said method comprising:
introducing a first stent graft through a first access site, the first stent graft comprising a proximal end, a distal end and an expandable cuff attached to the distal end;
introducing a second stent graft through a second access site, the second stent graft comprising a proximal end, a distal end and an expandable cuff attached to the distal end;
advancing the first and second stent grafts until at least a portion of each of the stent grafts extends across the deformity in the vessel wall; and
expanding the expandable cuffs of the first and second stent grafts to form a seal between the other of cuff and the vessel wall.

27. The method of claim 26 wherein the deformity is an aortic aneurysm and the first and second stent grafts are positioned by percutaneous delivery.

28. The method of claim 26 wherein the proximal end of the first stent graft remains in a first vessel while the proximal end of the second stent graft remains in a second vessel.

29. The method of claim 26 wherein the cuffs are inflatable and are inflated with a material comprising at least one of collagen and a mixture of thrombin and the patient's blood.

30. The method of claim 26 wherein each of the cuffs have a “D” configuration upon expansion and fill one half of the vessel while the stent grafts remain substantially circular in cross-section following deployment.

31. The method of claim 26 wherein each of the cuffs further include an inflation tube extending therefrom, said method further comprising inflating the cuffs with fluid passing through the inflation tube.

32. The method of claim 26 wherein the inflation tube is perforated, said method further comprising separating the inflation tube from the cuff along the perforation.

33. The method of claim 26 wherein the cuffs further include an inflation port having a valve configured to seal the cuff upon removal of an inflation tube, said method further comprising removing the inflation tube from the cuff after the cuff has been inflated such that the cuff remains sealed in the expanded configuration after the inflation tube has been removed.

34. The method of claim 26 wherein each said stent graft has a “D” shaped cross-sectional configuration including a flattened portion and an arcuate portion, each said stent graft also including a radiopaque marker, said method further comprising aligning said stent grafts using the radiopaque markers such that the flattened portions are adjacent each other.

35. A stent graft comprising:
first and second ends;
a first cuff located at said first end; and
a second cuff located at said second end, each said cuff configured to expand and exert a radially outward force against a vessel wall.

36. The stent graft of claim 35 wherein at least one said cuff comprises an inflation port including a valve configured to seal said cuff after an inflation tube is removed therefrom.

37. The stent graft of claim 35 where in at least one said cuff comprises an inflation tube extending therefrom, said inflation tube having a perforated section configured to be severed such that the distal end remains connected to said cuff after said inflation tube has been severed.

38. The stent graft of claim 35 wherein at least said cuff comprises a sponge material configured to expand upon exposure to moisture.

39. A stent graft comprising:
first and second ends; and
a first cuff located at said first end, said cuff configured to expand and exert a radially outward force against a vessel wall, said cuff comprising an inflation port including a valve configured to seal said cuff after an inflation tube is removed therefrom.

40. The stent graft of claim 39 wherein said cuff is configured to be inflated with a material that hardens upon expansion of said cuff.

41. The stent graft of claim 40 wherein the material comprises at least one of collagen and a mixture of thrombin and the patient's blood.

42. A stent graft comprising:
first and second ends; and
a first cuff located at said first end, said cuff configured to expand and exert a radially outward force against a vessel wall, said cuff comprising an inflation tube including a weakened portion that can be severed more easily than the remaining portion of said inflation tube.

43. The stent graft of claim 42 wherein said weakened portion is a perforated section configured to be severed such that a distal end of said inflation tube remains connected to said cuff after said inflation tube has been severed.

44. The stent graft of claim 42 wherein said cuff is configured to be inflated with a material that hardens upon expansion of said cuff.

45. The stent graft of claim 44 wherein the material comprises at least one of collagen and a mixture of thrombin and the patient's blood.

46. A stent graft comprising:
first and second ends; and
a first cuff located at said first end, said cuff configured to expand and exert a radially outward force against a vessel wall, said cuff comprising a sponge material configured to expand upon exposure to moisture.

47. The stent graft of claim 46 wherein the sponge material includes a thrombogenic material.

48. The stent graft of claim 46 wherein the sponge material includes a pro-coagulent.

49. The stent graft of claim 46 wherein said cuff is configured to harden upon exposure to blood after said cuff has expanded.
